(19) United States
(12) Patent Application Publication
(10) Pub. No.: US 2009/0018635 A1
(43) Pub. Date: Jan. 15, 2009

(54) STENT PROTECTOR

(75) Inventors: Thomas J. Holman, Minneapolis, MN (US); Tracee Eidenschink, Wayzata, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 11/775,340

(22) Filed: Jul. 10, 2007

(51) Int. Cl. A61F 2/84; B65D 69/00

(52) U.S. Cl. 623/1.11; 206/571

(57) ABSTRACT

A method for providing a catheter assembly with a protective sleeve for a stent delivery system wherein the protective sleeve has a removable portion that is removed from about the stent before the system is inserted into a patient, and the protective sleeve made thereby.

### FIELD OF THE INVENTION

The present invention relates to the field of expandable medical devices such as stents, including balloon expandable and self-expanding stents, and protectors therefore.

### BACKGROUND OF THE INVENTION

A stent, stent-graft or other expandable framework, is an implantable medical device introduced into a body lumen and is well known in the art. Typically, a stent is implanted in a blood vessel at the site of a stenosis or aneurysm endoluminally, i.e. by so-called “minimally invasive techniques” in which the stent, in a radially reduced configuration, optionally restrained in a radially compressed configuration by a sheath and/or catheter, is delivered by a stent delivery system or “introducer” to the site where it is required. The introducer can enter the body from an access location outside the body, such as through the patient's skin, or by a “cut down” technique in which the entry vessel is exposed by minor surgical means. The device is radially enlarged at the treatment site.

Stents can be implanted in a variety of body lumens or vessels such as within the vascular system, urinary tracts, bile ducts, fallopian tubes, coronary vessels, secondary vessels, etc. Stents can be self-expanding, expanded by an internal radial force, such as when mounted on a balloon, or a combination of self-expanding and balloon expandable (hybrid expandable).

Stent protectors are used to protect the stent before the stent and catheter assembly are introduced and subsequently the stent deployed and implanted into a body lumen. The stent protector protects the stent from physical damage or contamination due to the transfer of unwanted material and is removed at the time of use to permit deployment of the stent. Examples of stent protectors are provided in commonly assigned U.S. Pat. Nos. 6,991,639, 6,783,542, 6,764,504, 6,416,529, 6,152,944, 5,893,868, and 5,342,307, each of which is incorporated by reference herein in its entirety.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### SUMMARY OF THE INVENTION

The present invention relates to an improved stent protector and to methods of making and using the same. The improved stent protector reduces the possibility of damaging either the stent protector material on the inner surface of the stent protector and/or any stent coatings on the outer surface of a stent by frictional resistance resulting during deposition of the stent protector over a stent delivery assembly.

In one aspect, the present invention relates to a protector for a stent having a removable portion, the removable portion defined by annular perforations.

In another aspect, the present invention relates to a tubular protector for a stent, the stent having first and second end regions and a crimped state defined by a diameter D_{1b}, the tubular protector including a first state defined as having a diameter D_{1c} which is larger than D_{1b}, a second reduced state defined as having a diameter D_{2c} which is substantially equal to D_{1b} the protector further including annular perforations wherein at least a portion of the protector is removable and at least a portion of the protector is non-removable, the non-removable portion overlapping the first and second end regions of the stent.

In another aspect the present invention relates to a method of providing a catheter assembly with a protected configuration and a delivery configuration, the catheter comprising at least one shaft having a proximal end and a distal end, a stent having a length and first and second end regions, the stent having a crimped and at least one expanded configuration; the stent being disposed about the shaft, and a protective sleeve having a body portion and having first and second end regions. The method includes providing a tubular member, disposing the tubular member about the entire length of the stent in its crimped configuration, shrinking the tubular member about the stent to form the protected configuration and removing a portion of the protective sleeve leaving a remainder of the protective sleeve disposed over the first and second end regions of the stent to form the delivery configuration, wherein the remainder of the protective sleeve form stent retaining sleeves in the delivery configuration.

In another aspect, the present invention relates to a method for providing a catheter assembly with a stent protector, the method includes disposing the stent protector about the shaft/balloon/stent assembly in a tubular form, the tubular form having a diameter which is larger than that of the combined shaft/balloon/stent assembly, and then shrinking the tubular member into place, i.e. a shrink-wrapped stent protector.

The protector can then be annularly perforated so as to overlap with the proximal and distal end regions of the stent, leaving stent retaining sleeves disposed about the respective end regions of the stent, so that when the removable portion of the stent protector is removed, stent retaining sleeves are formed which just overlap the end regions of the stent, and the waist portions and cone portions, and the end regions of the body portion of the balloon for retention of the stent in place in a reduced configuration on the delivery catheter during stent delivery.

The protector may be designed so as to be completely removable or partially removable. For example, one of the proximal and/or distal ends can be removed, or suitable portions can be removed to provide for controlled stent deployment, such as for end up or center up deployment.

Perforation maybe accomplished either before of after disposal of the stent protector onto the assembly, i.e., in the tubular member, or in the formed stent protector.

Upon expansion of the balloon, the stent slips from the stent retaining sleeves. The balloon can then be deflated and withdrawn from the stent.

In an alternative design, the protector may be secured to the package which retains the catheter. Upon removal from the package, the protector stays with the package, and is automatically removed from the stent.

In another aspect, the present invention may be employed as a barrier for a stent having coatings disposed thereon which may be sensitive to the surrounding environment. For example, some stent coatings are moisture sensitive and it is desirable to protect these coatings from premature degradation during storage due to exposure to ambient moisture. The barrier may be removed from the stent prior to use.

The barrier may be shrunk during sterilization, and the ends mechanically sealed during this process.

In another aspect, the present invention relates to a catheter assembly disposed within a package. The catheter assembly includes a stent disposed about the distal end of an elongate catheter shaft and a protective sleeve disposed about the stent. The protective sleeve has a removable portion which is secured to an inner surface of the package. When the catheter assembly is removed from the package, the removable portion of the protective sleeve is removed from the catheter assembly. Any portion of the sleeve may be removable as described above. In one embodiment, the catheter assembly further includes an expandable balloon member, the expandable balloon member is disposed about the distal end of the elongate catheter shaft, the stent is disposed about the balloon, and the protective sleeve is disposed about both the balloon and the stent.

Any suitable adhesive which secures the protective sleeve to an inner surface of the package may be employed.

These and other aspects, embodiments and advantages of the present invention will become immediately apparent to those of ordinary skill in the art upon review of the Detailed Description and Claims to follow.

### BRIEF DESCRIPTION OF THE DRAWINGS

FIG. 1 is a partial side view showing the distal end of a catheter assembly with parts shown in hidden line.

FIG. 2 is a radial cross-section taken at section 2-2 in FIG. 1.

FIG. 3 is a partial side view showing the distal end of a catheter assembly with a tubular member disposed about the stent and balloon with parts shown in hidden line.

FIG. 4 is a partial side view showing the distal end of a catheter assembly after shrinking the tubular member shown in FIG. 3 about the stent and balloon with parts shown in hidden line

FIG. 5 is a partial side view showing the distal end of a catheter assembly with parts shown in hidden line after removal of a portion of the stent protector.

FIG. 6 is a side view of a carrier tube for a catheter assembly.

FIG. 7 is a longitudinal cross-section taken at section 7-7 in FIG. 6 showing a stent, balloon and protective sleeve disposed at the distal end of an elongate catheter shaft disposed within a carrier tube as shown in FIG. 6 and secured to the inner surface therein.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, unless otherwise indicated, identical reference numerals used in different figures refer to the same component. All US patents and applications and all other published documents mentioned anywhere in this application are incorporated herein by reference in their entirety.

The stent protectors according to the invention may be employed in combination with self-expanding and with balloon expandable stents.

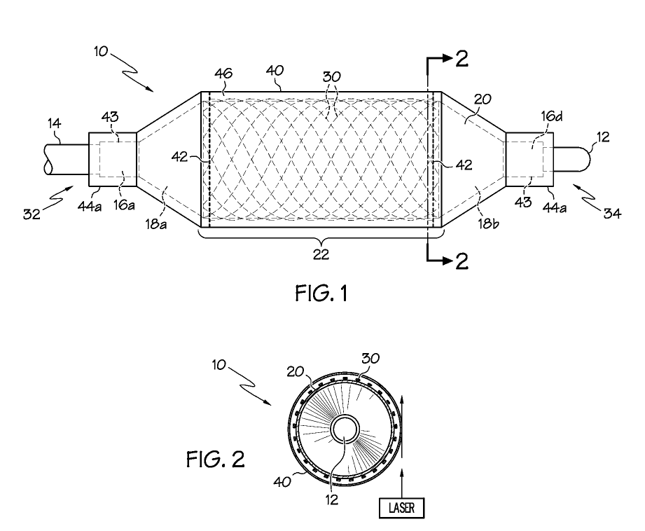

Turning now to the figures, FIG. 1 illustrates generally at 10, the distal end of a catheter assembly wherein stent protector 40 is employed in combination with a balloon expandable stent 30. In this embodiment, catheter assembly 10 has an inner shaft 12 and an outer shaft 14. The distal end of each shaft is shown in FIG. 1. Balloon 20 is disposed about the distal end of the outer shaft at the balloon proximal end 32 and is disposed about the distal end of the inner shaft 12 at the balloon distal end 34. Balloon 20 may be secured to the inner shaft 12 and the outer shaft 14 at its waist portions 16a, 16b using any suitable method known in the art such as welding, adhesively, etc.

Stent 30 is disposed about balloon 20 in the body region 22 of balloon 20. FIG. 2 is a radial cross-section taken at section 2-2 in FIG. 1.

Stent protector 40 is formed on the assembly by first disposing a tubular member 35 over balloon waist portions 16a, 16b, cone portions 18a, 18b and body portion 22 as shown in FIG. 3. The balloon 20 is in a folded/wrapped configuration and is defined by a diameter, D₁ₐ. The stent 30 is in a crimped state and is defined by a crimped diameter, D_{1b}. The tubular member is defined as having a diameter D_{1c} wherein D_{1c} is larger than the sum of D₁ₐ and D_{1b} during loading of the tubular member 35 onto the catheter assembly. Having a larger diameter prevents scraping of the inner surface of the tubular member 35 on the stent which can contaminate the stent coating, and prevents damage to the stent coating. As stent coatings often carry therapeutic agents, damage to the coating may adversely affect the drug delivery properties of the coating.

Suitably, the tubular member 35 is formed from a heat shrinkable polymer composition. Examples of polymers which may be employed in forming the heat shrinkable tubular member 35 are typically thermoplastic, although in some instances thermoset materials may be employed, and include both elastomeric and non-elastomeric polymer materials. Suitable examples include, but are not limited to, polyolefins including, for example, homopolymers, copolymers and terpolymers of ethylene and propylene, fluoropolymers such as fluorinated ethylene-propylene (FEP), polytetrafluoroethylene (PTFE), polyvinylidene fluorides (PVFD) such as Kynar® PVFD's including Kynar® 500 available from Arkema Inc. in Philadelphia, Pa., copolymers of hexafluoropropylene (HFP), terpolymers of tetrafluoroethylene (TFE), ethylene-chlorotrifluoroethylene (ECTFE), VDF and HFP as well as perfluoromethylvinylether (PMVE), Viton® fluoropolymer elastomers available from Du Pont Performance Elastomers in Wilmington, Del., polyvinyl chloride (PVC), neoprene, silicon elastomers, polyamides including the nylons, polyether-block-amides, etc. Blends of polymer materials as well as multilayer structures may be employed herein as well.

In preferred embodiments, the protective sleeve may be formed from a heat shrinkable material.

Preferred materials include, but are not limited to, homopolymers, copolymers and terpolymers of olefins, fluoropolymers, polyvinyl chloride (PVC), neoprene, silicon elastomers, and mixtures thereof.

The above list is intended for illustrative purposes only, and not as a limitation on the scope of the present invention.

The tubular member may be formed using any suitable method known in the art including, for example, extrusion or injection molding.

The tubular member can then be shrunk over the assembly by application of heat using an oven, hot air gun, or similar tool, causing the heat shrinkable tubular member 40 to contract to a second smaller diameter which is discussed in further detail below.

Temperatures used for shrinkage will depend on the polymer material selected for use. Many types of heat shrinkable materials have the ability to contract as far as one sixth the original diameter. Consequently, the size, i.e. diameter, of the protective sleeve 40 can be selected based on the diameter of the balloon/stent over which it is disposed, and on how much shrinkage is desirable to fit snuggly over the stent/balloon after shrinkage to provide the desirable snug fit over stent/balloon combination. As is known in the polymer art, if overheated, the heat shrinkable material can melt, scorch or catch fire.

Alternatively, the tubular member can be chemically expanded such as by swelling with a solvent. The tubular member then shrinks as the solvent evaporates. Polymer materials which may be employed for such an application may be lightly crosslinked polymer materials, and some thermoplastic materials.

It is desirable for the balloon 20 to be in a folded/wrapped state, and stent 30 is crimped onto balloon 20 in a reduced diameter configuration prior to shrinking of the protective sleeve as shown in FIG. 3. A balloon typically has a “static” state defined as being the diameter of the balloon as it is formed, i.e. as it comes out of a balloon mold, for example, prior to either inflation or deflation. Stent 30 has a nominal diameter prior to crimping to a reduced configuration or to expansion during deployment of the stent in a body vessel, the nominal diameter being greater than the crimped diameter, but less than the expanded diameter. As is known to those of skill in the art, a stent is not limited to one crimped state or one expansion state, but can have many.

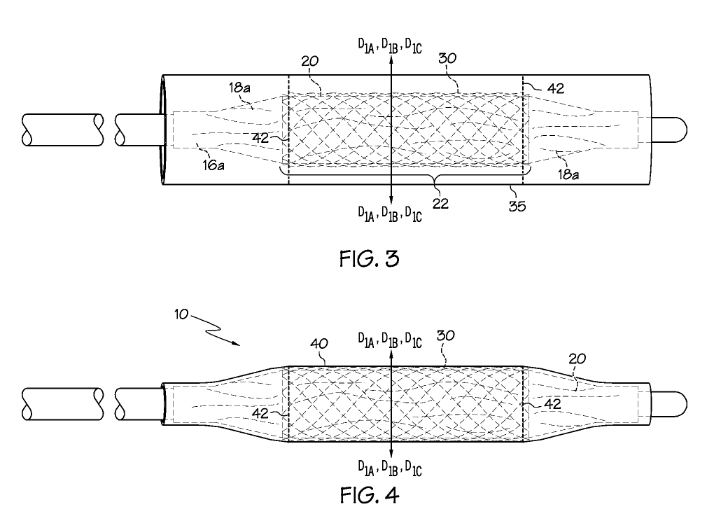

The diameter of the tubular member 35, D_{1c}, shall be defined herein as being the diameter of the tubular member 35 in its loading state while it is being disposed over the assembly (FIG. 3) and prior to shrinkage of the protective sleeve to a reduced diameter D_{2c} (FIG. 4). At the time of shrinking the tubular member 35 over the assembly, the balloon 20 is in a folded/wrapped state, and stent 30 is crimped onto the folded/wrapped balloon 20 as shown in FIG. 4 to a reduced diameter. For balloon folding, see commonly assigned U.S. Pat. No. 5,342,307 to Entenaur et al., the entire content of which is incorporated by reference herein. For balloon crimping prior to loading a protector, see commonly assigned U.S. Pat. No. 6,783,542 to Eidenschink, the entire content of which is incorporated by reference herein. The balloon 20 in FIG. 1 is not shown in a folded/wrapped configuration and stent 30 in FIG. 1 is not shown crimped.

Upon inflation of balloon 20 and expansion of stent 30 for deployment of stent 30 in a patient's body vessel, balloon 20 and stent 30 will also have a third enlarged diameter (not shown), larger than either the static state diameter or reduced configuration diameter of the balloon 20 and stent 30 as discussed above.

Suitably, at least a portion of the stent protector 40 is removable from the assembly prior to delivery and deployment of the stent. It is advantageous if a practitioner can readily remove the stent protector at the time of use. In one embodiment, at least a portion of the stent protector is removable via the use of small cuts or perforations 42 annularly spaced about each end of the body portion 46 of the stent protector 40 for creating a cut-path as shown in FIGS. 1, 3 and 4. The portion in between the cut-paths is removed prior to use. The stent protector may be totally removed or only partially removed. This flexibility allows for controlled stent deployment such as for center-up or end-up stent deployment.

The perforations may be either formed in the tubular member 35 as shown in FIG. 3, or they may be formed in the protective sleeve 40 as shown in FIGS. 1 and 4. In these embodiments, the perforations 42 may be located just over the end regions of stent 30 as shown in FIGS. 1, 3 and 4 as will be explained more fully below.

Any suitable method may be employed for formation of the perforations 42 including laser energy. The perforations 42 may be created in the stent protector for creation of an annular cut-path in the stent protector either before placement over the catheter assembly, or after placement over the catheter assembly. FIG. 2 illustrates application of laser energy for formation of the perforations 42 in the stent protector 40 after disposal over the catheter assembly 10. Suitably, this is accomplished after the stent protector 40 has been shrunk over the assembly 10.

Any suitable laser may be employed for formation of the perforations 42 including, for example, UV Excimer lasers and Nd:Yag lasers. Other cutting methods include, but are not limited to, the use of razor blades, stamping dies, etc.

It may be desirable to secure the waist portions 44a, 44b of the stent protector 40 to the corresponding waist portions 16a, 16b of the balloon 20. This can be accomplished by laser welding techniques, for example.

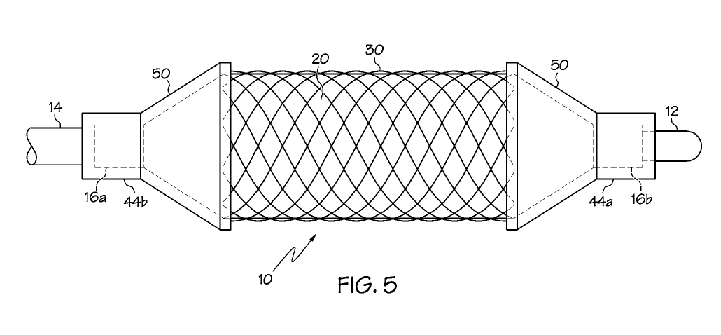

At the time of use, the practitioner can then remove the portion 46 of the stent protector 40 located between the perforations 42 or cut-paths as shown in FIG. 5. In this embodiment, the portion removed is designed so as to leave stent retaining sleeves or sox 50 for retention of the stent 30 during delivery through a patient's vasculature. Upon expansion of the balloon 20 and consequently stent 30, the stent 30 slips from retaining sleeves 50, which have been secured at the waist portions 44a, 44b to the corresponding waist portions 16a, 16b of balloon 20 thereby deploying stent 30a. The balloon 20 can then be deflated along with retaining sleeves 50. It is surmised that the retaining sleeves 50 actually facilitate balloon deflation and rewrap. Balloon 20 and sleeves can then be withdrawn from the patient's body.

In one embodiment, the removable portion 46 of the stent protector 40 may be secured to an inner wall of a package so that when the catheter assembly is removed from the package, the removable portion 46 remains with the package. This facilitates removal of the removable portion 46 of the stent protector 40 without an additional step on the part of the medical practitioner.

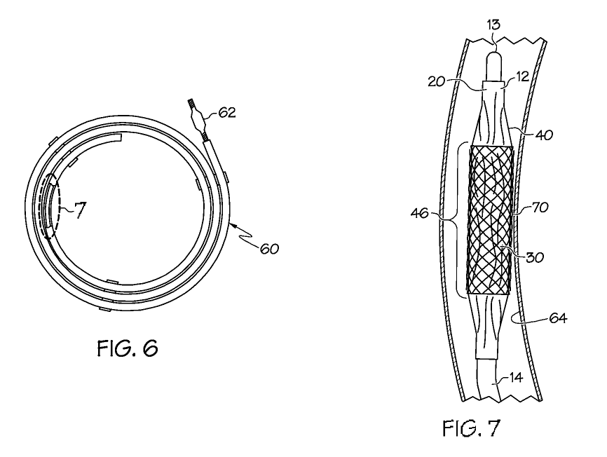

FIG. 6 is a side view of one embodiment of a catheter package otherwise referred to herein as a carrier tube 60, within which a catheter assembly may be disposed. One example of this type of catheter packaging is disclosed in commonly assigned copending U.S. Patent Publication No. 2003/0125713, the entire content of which is incorporated by reference herein. Discernable from FIG. 7, is the hub assembly 62 which is disposed at the proximal end of the catheter assembly (not shown). FIG. 7 is a longitudinal cross-section of the carrier tube 60 taken at section 7-7 in FIG. 6. The distal end of the catheter assembly with balloon 20, stent 30 and stent protector 40 disposed thereon, is shown within the carrier tube. The removable portion 46 of the stent protector 40 is shown secured to the inner wall 64 of carrier tube 60 using any suitable method of securement known in the art, including both adhesive as well as mechanical securement. Mechanical securement may be accomplished via laser welding, for example.

The present invention may also be employed as a barrier for a stent having a coating disposed thereon. For example, some coatings are moisture sensitive and it may be desirable to protect such coatings from premature degradation due to exposure to ambient moisture during storage.

For example, the coating may include bioresorbable polymers. Examples of bioresorbable polymers include, but are not limited to, poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen, hyaluronic acid, etc., and mixtures thereof.

The present invention may also be employed wherein hydrophilic coatings such as hydrogel coatings are disposed on the stent.

Any of the coatings may incorporate a therapeutic agent therein. The terms, “therapeutic agent”, “drug”, “pharmaceutically active agent”, “pharmaceutically active material”, “beneficial agent”, “bioactive agent”, and other related terms may be used interchangeably herein and include genetic therapeutic agents, non-genetic therapeutic agents and cells. A drug may be used singly or in combination with other drugs. Drugs include genetic materials, non-genetic materials, and cells.

The barrier can also prevent premature elution of the therapeutic agent from the coating. Examples of suitable drugs can be found in commonly assigned U.S. Pat. No. 7,105,175, 7,014,654, 6,899,731, 6,855,770 and 6,545,097, each of which is incorporated by reference herein in its entirety, and in commonly assigned U.S. Patent Application Publication No. 2004/0215169, the entire content of which is incorporated by reference herein.

The above lists are intended for illustrative purposes only, and not as a limitation on the scope of the present invention.

In an embodiment wherein the present invention is employed as a barrier, the ends are suitably mechanically sealed. This can be accomplished by heat shrinking the tubular member during the sterilization process.

In another embodiment, the present invention is employed as a drug eluting layer. In this embodiment, the central portion of the tubular member is left on, while the proximal and distal ends are removed. The central portion contains therapeutic agent therein.

Methods of making the stent protectors are also contemplated herein. In one embodiment, a method of providing a catheter assembly with a protected configuration and a delivery configuration is contemplated.

The method may be directed to stent protectors for self-expanding stents, as well as stent protectors for balloon expandable stents. In one embodiment, the catheter includes a balloon having a body, cone and waist portions, the body having first and second end regions, a stent having a length and first and second end regions, the stent being disposed about at least a portion of the body of the balloon, and a protective sleeve having a body portion having first and second end regions. The method includes providing a tubular member, disposing the tubular member about the body, cone and waist portions of the balloon and the entire length of the stent, shrinking the tubular member about the body, cone and waist portions of the balloon and the entire length of the stent to form the protected configuration and removing a portion of the protective sleeve from the body portion of the balloon leaving a remainder of the protective sleeve disposed over the waist and cone portions, and the first and second end regions of the balloon body, and disposed over the first and second end regions of the stent to form the delivery configuration, wherein the remainder of the protective sleeve forms stent retaining sleeves in the delivery configuration.

Perforations or other slits may be provided in the protective sleeve. Suitably, the perforations are spaced annularly about the protective sleeve and overlap the first and second end regions of the stent, and the removable portion of said protective sleeve is disposed between the annular perforations.

The perforations may be provided in the protector using any suitable method known in the art. One method of providing the perforations is to use a laser. UV Excimer lasers or Nd:Yag lasers may be employed.

The protective sleeve may have a loading configuration as well. In the loading configuration, the protective sleeve has a first diameter, D_{1c} and in the protected configuration has a diameter D_{2c} which is less than D_{1c} wherein D_{1c} and D_{2c} are determined at a central region of the protective sleeve. In one embodiment, the protective sleeve in its loading configuration has a diameter D_{1c}, the balloon is in a folded state having a diameter D₁ₐ and the stent is in a crimped state having a diameter D_{1b} and is crimped onto the body portion of the stent wherein D_{1c} is greater than the sum of D₁ₐ and D_{1b}.

In another embodiment, the protected configuration of the protective sleeve is defined by a diameter D_{2c} wherein D_{2c} is substantially equal the sum of D₁ₐ and D_{1b}.

The protective sleeve may be secured at the waist portions of the balloon. Securement may be accomplished in the delivery configuration. In embodiments where the stent is self-expanding, the protective sleeve may be secured a catheter shaft.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

1. A method for providing a catheter assembly with a stent protector at least a portion of which is removable, the catheter assembly comprising a balloon having a body, cone and waist portions, the body having first and second end regions; a stent having a length and first and second end regions, the stent being disposed about at least a portion of the body of the balloon, the balloon in a folded state defined as having a diameter D₁ₐ and the stent in a crimped state defined as having a diameter D_{1b}, the stent crimped over the body portion of the balloon, the method comprising:
providing a tubular member having a diameter, D_{1c} which is larger than the sum of the diameters D₁ₐ and D_{1b},
disposing said tubular member over said balloon and said stent;
shrinking said tubular member, wherein said tubular member is disposed over the body, cone and waist portions of the balloon and over the entire length of said stent to form a protective sleeve having a diameter D2_{c} which is substantially equal to D₁ₐ and D_{1b}, wherein at least a portion of said protective sleeve is removable and at least a portion of said protective sleeve is non-removable, the non-removable portion overlapping the first and second end regions of said stent, and overlapping said waist portions, cone portions and the end regions of the body portion of the balloon; and
removing said removable portion of said protective sleeve prior to insertion in a body lumen.

2. The method of claim 1 wherein shrinking is conducted by applying heat.

3. The method of claim 1 further comprising swelling said tubular member in solvent.

4. The method of claim 3 further comprising sterilizing said assembly, wherein said shrinking of said tubular member is simultaneous with sterilizing said tubular member.

5. The method of claim 1 further comprising securing said protective sleeve to said balloon at said waist portions of said balloon.

6. The method of claim 1 further comprising providing perforations spaced annularly about said tubular member, wherein when said tubular member is disposed over said balloon and said stent, said perforations overlap said first and second end regions of said stent.

7. The method of claim 6 further said perforations are provided in said protective sleeve is after heat shrinking said protective sleeve.

8. The method of claim 1 wherein said perforations are provided in said protective sleeve using a laser.

9. The method of claim 1 further comprising forming said tubular member.

10. The method of claim 9 wherein said tubular member is formed from a polymer composition, the polymer composition comprising at least one member selected from the group consisting of homopolymers, copolymers and terpolymers of olefins, fluoropolymers, polyvinyl chloride (PVC), neoprene, silicon elastomers, and mixtures thereof.

11. The method of claim 1 further comprising disposing said catheter assembly in a package, the package having an inner surface and an outer surface, the package further comprising an adhesive on said inner surface, the adhesive securing said removable portion of said protective sleeve to said package.

12. The method of claim 12 further comprising removing said catheter assembly from said package, and said removing of said removable portion is substantially simultaneous with said removing said catheter assembly from said package.

13. A method of providing a catheter assembly with a protected configuration and a delivery configuration, the catheter comprising at least one shaft having a proximal end and a distal end, a stent having a length and first and second end regions, the stent having a crimped and at least one expanded configuration; the stent being disposed about the shaft, and a protective sleeve having a body portion and having first and second end regions, the method comprising:
providing a tubular member;
disposing said tubular member about the entire length of the stent in its crimped configuration;
shrinking said tubular member about said stent to form said protected configuration; and
removing a portion of said protective sleeve leaving a remainder of said protective sleeve disposed over the first and second end regions of the stent to form said delivery configuration, wherein the remainder of said protective sleeve forms stent retaining sleeves in said delivery configuration.

14. A combination of a catheter assembly and a package, the combination comprising:
an elongate catheter shaft comprising a proximal end and a distal end:
a stent disposed about the distal end of the catheter shaft;
a protective sleeve disposed about the stent, the protective sleeve comprising a removable portion;
a package having an inner surface and an outer surface, the catheter assembly disposed within the package; and
wherein the removable portion of the protective sleeve is secured to the inner surface of the package.

15. The combination of claim 14 further comprising an expandable balloon member, the expandable balloon member disposed about the distal end of the catheter shaft, the stent disposed on the expandable balloon member, the protective sleeve disposed about the stent and the expandable balloon member.

16. The combination of claim 14 wherein said package is a carrier tube.

17. The combination of claim 14 wherein said removable portion of said protective sleeve is secured to the inner surface of the package adhesively or by welding.

18. A protector for a stent comprising a removable portion, the removable portion defined by annular perforations.

19. The protector of claim 18 comprising first and second end regions, the first and second end regions are non-removable.

20. The protector of claim 19, said stent having first and second end regions, the first and second end regions of the protector over lap the first and second end regions of the stent.

21. The protector of claim 19 wherein said stent is disposed about the distal end of a catheter assembly, the catheter assembly comprising at least one shaft, the first and second end regions of said protector further overlap said shaft and are secured thereto.

22. The protector of claim 19 further in combination with an expandable medical balloon, the balloon having body portion, first and second waist portions and first and second cone portions, the balloon disposed about a catheter shaft, the first and second end regions of said protector overlap said first and second waist portions of said balloon and are secured thereto.

23. The protector of claim 189 formed from a heat shrinkable material.

24. A tubular protector for a stent, the stent having first and second end regions and a crimped state defined by a diameter D_{1b}, the tubular protector comprising:
a first state defined as having a diameter D_{1c} which is larger than D_{1b},
a second reduced state defined as having a diameter D_{2c} which is substantially equal to D_{1b}
the protector further comprising annular perforations wherein at least a portion of said protector is removable and at least a portion of said protector is non-removable, the non-removable portion overlapping the first and second end regions of said stent.
